(19) United States
(12) Patent Application Publication
(10) Pub. No.: US 2003/0022394 A1
(43) Pub. Date: Jan. 30, 2003

(54) Biochips and methods of making same

(76) Inventor: David P. Dumas, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/205,745

(22) Filed: Jul. 26, 2002

Related U.S. Application Data
(60) Provisional application No. 60/308,973, filed on Jul. 27, 2001.

(51) Int. Cl. G01N 1/00; G01N 33/53; G01N 33/543; B05D 3/00; B32B 5/02

(52) U.S. Cl. 436/518; 436/174; 422/82.11; 435/7.1; 427/2.11

(57) ABSTRACT

The invention provides a method of generating an array of molecules. The method includes the steps of attaching one or more chemical moieties to a plurality of waveguides; distributing the plurality of waveguides in a predetermined arrangement in a grid, the grid thereby spacing the waveguides at a predetermined distance; embedding the plurality of waveguides in an inert support; and slicing the embedded plurality of waveguides transversely, thereby generating an array of molecules. A non-end portion of the embedded waveguides can be removed from at least one face of the slice. The waveguides can also be coated with cladding to effect spacing at a predetermined distance. The invention also provides compositions comprising a plurality of waveguides embedded in an inert support, the waveguides having attached thereto one or more chemical moieties, having a non-end portion exposed, and being spaced at a distance to minimize crosstalk between waveguides.

This application claims benefit of the filing date of U.S. Provisional Application No. 60/308,973, filed Jul. 27, 2001, and which is incorporated herein by reference.

The present invention relates generally to genomics and proteomics and more specifically to biochips useful in genomics and proteomics analysis.

### BACKGROUND OF THE INVENTION

Greater than 300,000 different proteins are estimated to be present in humans. Of these proteins, there are about 15,000 potential molecular therapeutic targets. To date, less than 1000 have been identified and exploited for pharmaceuticals. In an attempt to identify which of the remaining 299,000 proteins are viable pharmacological targets, various genomic tools have been developed to analyze anomalies in the genetic code or mRNA levels.

Genomics has been developed over the last decade in part to identify new targets and has led to the development of new diagnostic methods. Leads identified by changes in mRNA levels have fueled the high throughput screening groups of the major pharmaceutical companies, many of which screen as many as 100 targets per year.

In addition to genomics analysis, the field of proteomics was developed. Rather than measuring the expression of genes as mRNA, proteomics measures protein expression levels, including changes in post-translational modification of proteins. Both the field of genomics and proteomics have utilized array technology to determine mRNA and protein expression.

A variety of technologies have developed over the past few years to determine gene and protein expression for genomics and proteomics analysis, respectively. For example, the use of photolithography has allowed the production of high-density biosensor arrays (U.S. Pat. No. 5,405,783). Each array produced in this method requires multiple series of light activation with multiple chemical steps. Each instance of light activation requires an expensive lithography mask that is subject to deterioration or damage during use. The high cost of production equipment, manufacture, and analytical instrumentation has hindered the broad application of biochip technology to diagnostics, drug discovery, and basic research.

Bundles of optical fibers have been proposed as an option for the production of biosensor devices (Anderson et al., Proceedings SPIE—The Intl. Soc. for Optical Engineering 1648:39-43 (1992); U.S. Pat. No. 5,790,727; U.S. Pat. No. 6,137,117; WO9858079). Optical fibers or waveguides were used to which organic molecules are covalently attached to the sensor ends. A biosensor array of optical fibers bundled together has been described (U.S. Pat. No. 6,146,593). Fibers with a DNA molecules covalently attached to each of the ends of the fibers are combined at their sensor ends to produce a high density sensor array of fibers capable of assaying simultaneously the binding of components of a test sample to the various binding partners on the different fibers of the sensor array. The transmission ends of the optical fibers are then discretely addressed to detectors such as optical sensors. An optical signal, produced by binding of the binding partner to its substrate to form a binding complex, is conducted through the optical fiber or group of fibers to a detector for each discrete test.

It has been recognized that a multitude of biosensor devices can be manufactured using a similar approach where functional moieties are synthesized onto fibers. Once the functional moieties are synthesized onto the fibers, the fibers are bundled in a predetermined arrangement. The bundled fibers are then bonded with a chemical adhesive to fix their predetermined arrangement. Finally, the bonded fiber bundle is “sliced” into a biochips (U.S. Pat. No. 6,129,896). Although this type of method for producing biochips circumvents the need for sophisticated photolithography instrumentation, bundling of the fibers would cause a significant loss in signal due to crosstalk between adjacent fibers. Furthermore, the use of a chemical adhesive or glass fusion to bundle fibers, as described in U.S. Pat. No. 6,129,896, would lead to the degradation of surface biomolecules. In addition, the surface area available to interact with a biological sample using this method would be a 2-diminsional face of the cut fiber, which has the disadvantage that the surface attached biomolecules are minimally exposed around the circumference of each fiber.

Thus, there exists a need to develop cost effective methods for the production of high-density biochips. The present invention satisfies this need and provides related advantages as well.

### SUMMARY OF THE INVENTION

The invention provides a method of generating an array of molecules. The method includes the steps of attaching one or more chemical moieties to a plurality of waveguides; distributing the plurality of waveguides in a predetermined arrangement in a grid, the grid thereby spacing the waveguides at a predetermined distance; embedding the plurality of waveguides in an inert support; and slicing the embedded plurality of waveguides transversely, thereby generating an array of molecules. A non-end portion of the embedded waveguides can be removed from at least one face of the slice. The waveguides can also be coated with cladding to effect spacing at a predetermined distance. The invention also provides compositions comprising a plurality of waveguides embedded in an inert support, the waveguides having attached thereto one or more chemical moieties, having a non-end portion exposed, and being spaced at a distance to minimize crosstalk between waveguides.

### BRIEF DESCRIPTION OF THE DRAWINGS

FIG. 1 is a schematic diagram showing the synthesis of oligonucleotides on the surface of fibers.

FIG. 2 is a schematic diagram showing the alignment of fibers in an array.

FIG. 3 is a schematic diagram showing the transverse cutting of a fiber array into a plurality of biochips.

FIG. 4 shows the dissolution of the biochip surface, resulting in the exposure of the fiber ends.

FIG. 5 is a schematic diagram showing that, to minimize crosstalk between two fibers, the spacing between fibers is dependent upon the height of the exposed fiber (h), the radius of the fiber (r), and the critical angle of the fiber (f).

### DETAILED DESCRIPTION OF THE INVENTION

The present invention provides methods of generating arrays of molecules that can be used as biochips. The biochips can serve as a biosensor device, which can be useful for separation and detection of small quantities of proteins, nucleic acids and organic molecules. The invention also provides methods of generating such biochips, which can be implemented as microarrays that can be used for high throughput screening applications, bioremediation and detection and quantification.

The methods of the invention can be used to produce a multitude of biosensor devices from a plurality of waveguides or optical fibers, where a discrete optical signal on a single waveguide or optical fiber is isolated from other waveguides or optical fibers. Furthermore, the methods of the invention can be used to produce a multitude of biosensor devices with improved sensitivity over 2-dimensional arrays. The present invention provides a method to incorporate a plurality of optical fibers with surface attached molecules for the production of a plurality of biosensor devices. Thus, the disclosed invention provides a significant improvement over previously described methods in providing a cost effective, quality determinative biosensor device and method of its manufacture.

As used herein, an “array of molecules” refers to a solid support having molecules distributed thereon in a fixed geometric arrangement.

As used herein, a “waveguide” is a material medium that confines and guides a propagating electromagnetic wave. In optical applications, a waveguide consists of a solid dielectric filament such as an optical fiber. An optical filament is a transparent dielectric material, for example, glass or plastic, that guides light. An optical fiber can be circular in cross section.

As used herein, the term “polypeptide” refers to a peptide, polypeptide or protein of two or more amino acids. A polypeptide can also be modified by naturally occurring modifications such as post-translational modifications or synthetic modifications, including phosphorylation, lipidation, prenylation, sulfation, hydroxylation, acetylation, addition of carbohydrate, addition of prosthetic groups or cofactors, formation of disulfide bonds, proteolysis, assembly into macromolecular complexes, and the like.

A modification of a peptide can also include non-naturally occurring derivatives, analogues and functional mimetics thereof generated by chemical synthesis. Derivatives can include chemical modifications of the polypeptide such as alkylation, acylation, carbamylation, iodination, or any modification that derivatizes the polypeptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine. Also included as derivatives or analogues are those polypeptides which contain one or more naturally occurring amino acid derivatives of the twenty standard amino acids, for example, 4-hydroxyproline, 5-hydroxylysine, 3-methylhistidine, homoserine, ornithine or carboxyglutamate, and can include amino acids that are not linked by peptide bonds.

As used herein, the term “nucleic acid” or “oligonucleotide” means a polynucleotide such as deoxyribonucleic acid (DNA) or ribonucleic acid (RNA). The terms include modified forms of naturally occurring nucleotides, so long as the nucleic acid or oligonucleotide retains a desired function.

As used herein, the term “antibody” is used in its broadest sense to include polyclonal and monoclonal antibodies, as well as antigen binding fragments of such antibodies. An antibody useful in the invention, or antigen binding fragment of such an antibody, is characterized by having specific binding activity for a ligand or sample epitope of at least about 1×10⁵ M⁻¹. Thus, Fab, F(ab′)₂, Fd, Fv, single chain Fv (scFv) fragments of an antibody and the like, which retain specific binding activity for a ligand, are included within the definition of an antibody. Specific binding activity of an antibody for a ligand can be readily determined by one skilled in the art, for example, by comparing the binding activity of an antibody to a particular ligand versus a control ligand that differs from the particular ligand. Specific binding can similarly be determined for a binding molecule for the ligand that is not an antibody. Methods of preparing polyclonal or monoclonal antibodies are well known to those skilled in the art (see, for example, Harlow and Lane, Antibodies: A Laboratory Manual, Cold Spring Harbor Laboratory Press (1988)).

In addition, the term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains as described by Huse et al. (Science 246:1275-1281 (1989)). These and other methods of making functional antibodies are well known to those skilled in the art (Winter and Harris, Immunol. Today 14:243-246 (1993); Ward et al., Nature 341:544-546 (1989); Harlow and Lane, supra, 1988); Hilyard et al., Protein Engineering: A practical approach (IRL Press 1992); Borrabeck, Antibody Engineerinq, 2d ed. (Oxford University Press 1995)). A particularly useful method for generating antibodies is based on using combinatorial libraries consisting of variable heavy chains and variable light chains (Kang et al., Proc. Natl. Acad. Sci. USA, 88:4363-4366 (1991), Huse et al., Science 246:1275-1281 (1989)).

The invention provides a method of generating an array of molecules. The method includes the steps of attaching one or more chemical moieties to a plurality of waveguides; distributing the plurality of waveguides in a predetermined arrangement in a grid, the grid thereby spacing the waveguides at a predetermined distance; embedding the plurality of waveguides in an inert support; and slicing the embedded plurality of waveguides transversely, thereby generating an array of molecules. The method can further comprise the step of removing a portion of the embedding inert support to expose a non-end portion of the embedded waveguides of at least one face of the slice.

The methods of the invention can be advantageously used to provide sensors that can be manufactured on an extremely low cost basis. The improved biosensor devices are manufactured by first synthesizing or attaching a plurality of molecules onto a plurality of fibers such as a waveguide or optical fiber, and the biosensor can be generated so that each fiber receives one specific type of molecule, if desired. The methods of the invention can be used to generate arrays on waveguides or optical fibers that allow light based assays to be used to characterize binding to the array. The methods disclosed herein can also be used with non-optical fibers, depending on the needs of the user. It is understood that methods described herein using waveguides or optical fibers can similarly be performed using non-optical fibers. As used herein, a fiber is intended to include a non-optical fiber as well as a waveguide or optical fiber.

Once the molecules are attached to the fibers, the fibers can be aligned in a predetermined array pattern using spacers to insure optimized separation between each fiber and optical isolation of each fiber. The fiber array is then embedded in an inert support to fix the position of the fibers. The inert support can be, for example, plastic or other non-covalently bonding support matrix. The formed fiber array is transversely cut into a plurality of devices (chips). On one face of the chip, the fiber ends can be exposed by removing a portion of the support matrix, for example, by dissolution in a solvent or by exposure to heat. The fibers can be glass fibers, for example, glass fibers suitable as optical fibers.

The methods of the invention are useful for generating replicate arrays of molecules, where any of a variety of desired molecules can be attached to or synthesized on the fibers such as a waveguide or optical fiber. For example, a chemical moiety can be attached to or synthesized on a fiber, for example, a nucleotide, nucleic acid such as oligonucleotide, amino acid, polypeptide, or antibody. Generally, an individual fiber will contain multiple copies of the same molecule, with different molecules attached separately to a plurality of fibers. For example, peptides or oligonucleotides can be attached to the waveguides or optical fibers. Methods for coupling molecules to a solid support are well known to those skilled in the art depending on available reactive groups on the fibers as well as the reactive group on the chemical moieties to be attached (see, for example, Glazer et al., Chemical Modification of Proteins. Selected Methods and Analytical Procedures, Elsevier Biomedical Press, New York (1975); Pierce Chemical Co.; Rockford Ill.). As described below, the fibers can be modified to incorporate a chemical functionality suitable for coupling to chemical moieties.

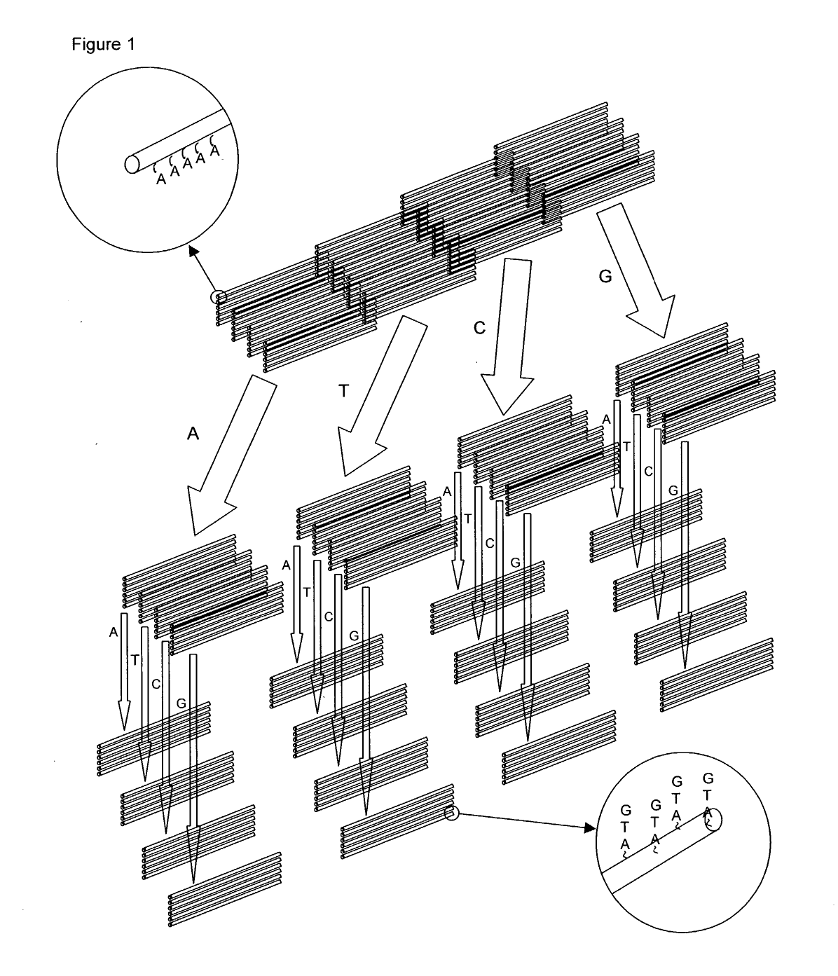

Alternatively, peptides or oligonucleotides can be synthesized by the sequential addition of amino acids or nucleotides, as illustrated in FIG. 1. Methods for peptide synthesis and the production of peptide libraries are well known to those skilled in the art (see, for example, Fodor et. al., Science 251:767 (1991); Gallop et al., J. Med. Chem. 37:1233-1251 (1994); Gordon et al., J. Med. Chem. 37:1385-1401 (1994)). Methods of oligonucleotide synthesis are also well known to those skilled in the art (see, for example, Ausubel et al., Current Protocols in Molecular Biology (Supplement 54), John Wiley & Sons, New York (2001)).

In addition, other types of organic molecules that are not naturally occurring biomolecules such as naturally occurring amino acids, polypeptides, nucleotides, nucleic acids or antibodies can also be attached to or synthesized on the fibers. For example, combinatorial chemical libraries can be attached to or synthesized on the fibers, if desired. Methods for synthesizing chemical compounds, including combinatorial chemical libraries, where the chemical compounds can be synthesized on solid phase, are well known to those skilled in the art (see, for example, Mendonca and Xiao, Med. Res. Rev. 19:451-462 (1999); van Maarseveen, Comb. Chem. High Throughput Screen. 1:185-214 (1998); Andres et al., Comb. Chem. High Throughput Screen. 2:191-210 (1999); Sucholeiki, Mol. Divers. 4:25-30 (1998-1999); Ito and Manabe, Curr. Opin. Chem. Biol. 2:701-708 (1998); Labadie, Curr. Opin. Chem. Biol. 2:346-352 (1998); Backes and Ellman, Curr. Opin. Chem. Biol. 1:86-93 (1997); Kihlberg et al., Methods Enzymol. 289:221-245 (1997); Blackburn and Kates, Methods Enzymol. 289:175-198 (1997); Meldal, Methods Enzymol. 289:83-104 (1997); Merrifield, Methods Enzymol. 289:3-13 (1997); Thuong and Asseline, Biochimie. 67:673-684 (1985)).

Fibers such as waveguides or optical fibers can be readily derivitized to allow the synthesis of oligonucleotides, peptides, or other molecules on the surface of the fiber using, for example, aminosiloxane to generate reactive amino functionalities. Methods for generating hydroxyl groups on glass fibers has been described (see, for example, U.S. Pat. No. 6,129,896). Other methods of generating reactive functionalitites on the surface of the fibers are readily known to those skilled in the art (see, for example, U.S. Pat. No. 5,419,966; U.S. Pat. No. 5,037,882; Nelson et al., Nucleic Acids Res. 17:7187-7194 (1989); Reed et al., Bioconjugate Chem. 2:217-225 (1991); Pon et al., BioTechnipues 6:768-775 (1988); Atkinson et al., in Oligonucleotide Synthesis: A Practical Approach, Gait, ed., pp. 35-81, IRL Press (1984)).

An example of attaching one or more chemical moieties to fibers by chemical synthesis is illustrated in FIG. 1. Individual nucleotides are coupled separately to a plurality of fibers (the coupling of A is illustrated in FIG. 1). The plurality of fibers are subdivided into four groups that are in turn reacted to couple another nucleotide, adenosine (A), guanosine (G), thymidine (T), and cytidine (C). Repetitive division of each group of fibers after addition of a nucleotide followed by coupling of nucleotides allows the synthesis of any sequence of nucleotides on the surface of the fibers.

The method of synthesizing a chemical moiety on a plurality of fibers can be used advantageously to minimize the number of chemical steps by reacting multiple groups of fibers together. For instance, 1600 fibers can be divided into 4 equal groups of 400 fibers. Each group of fibers (labeled A, T, C, or G in FIG. 1) is reacted to couple one of the four bases. Groups A, T, C, and G are then each subdivided into four equivalent subgroups of 100 fibers each. One group from each of the A, T, C, and G subgroups is labeled and reacted together to couple A to each of the fibers to generate the sequences AA, TA, CA, and GA. Similarly, A, T, C, and G fibers are reacted with the other bases to generate AT, TT, CT, GT, AC, TC, CC, GC, AG, TG, CG, and GG. These 16 different dinucleotides would be generated with only eight reactions. Continuing this method of dividing fiber groups, labeling, and combining with other fiber groups allows the synthesis of all 64 possible trinucleotides in a total of 12 reactions, all 256 tetranucleotides in 16 reactions, and all 1024 pentanucleotides in 20 reactions. This combinatorial method allows the synthesis of 4ⁿ different oligonucleotides with only 4n steps, where n is the number of nucleotides in the sequence. Multiple peptide sequences and other combinatorial libraries can be similarly synthesized on the fibers. Unlike other biochip manufacture methods, quality control of the individual fibers is allowed by standard analytical methods using short pieces removed from the ends of the fiber.

Thus, the invention provides a method of generating an array of molecules by distributing a plurality of waveguides, the waveguides having one or more chemical moieties attached thereto, in a predetermined arrangement in a grid, the grid thereby spacing the waveguides at a predetermined distance; and embedding said plurality of waveguides in an inert support. The method can further include the step of slicing the embedded plurality of waveguides transversely, thereby generating an array of molecules.

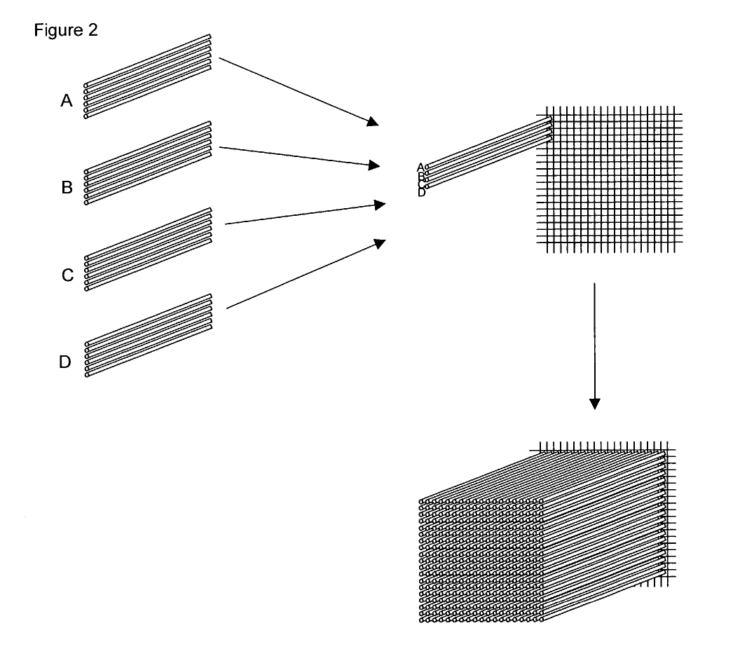

Individual fibers on which a chemical moiety has been synthesized or attached are distributed in a predetermined arrangement at a predetermined distance. For example, the fibers can be distributed in a grid to form an array, as illustrated in FIG. 2. Alternatively, the fibers can be spaced at a predetermined distance by coating the fibers with cladding, as described in more detail below. One method of generating the array with a grid involves inserting fibers through a wire mesh or screen, as shown in FIG. 2. Other methods of spacing fibers in arrays can utilize plastic, metal, glass, or fiber spacers to form a grid. The grid can be a preformed grid such as that shown in FIG. 2. Alternatively, the grid can be formed in layers as the fibers are distributed in the grid. For example, a first layer of grid can have fibers distributed in the grid and then overlayed with another layer of grid and fibers, and so forth. If desired, a grid can be placed at both ends of the fibers, or multiple grids can be placed along the length of the fibers, to provide a predetermined distance between the fibers along the entire length of the fibers (see Example I). The number of grids can be varied depending on the length and flexibility of the fibers. The openings in the grid can be designed to accommodate a single fiber, if desired, so that only a single fiber will occur at a particular position. Although different molecules are generally attached to separate fibers, the array can include replicates of the same fiber with the same attached molecules, which can be useful for quantitative assays.

The fiber array is then embedded in an inert support, for example, a plastic support. An inert support is an embedding material that does not substantially alter the desired function of the chemical moieties attached to the fibers. For example, in the case of oligonucleotide chemical moieties, the desired function can be hybridization to complementary nucleic acids. As such, an inert support is one which does not alter the chemical functionalities of the oligonucleotides in a manner that prevents hybridization. Similarly, an inert support with respect to an attached polypeptide or antibody is one that does not substantially alter the binding properties of the attached molecules. Particularly useful plastics for an inert support include polycarbonates, polystyrene, polypropylene, acrylonitrile-butadiene-styrene (ABS), acrylates, polymethylpentene, polyetherimide, polyphenylene oxide, polyvinylidene fluoride, acetal (Ultraform®), Hytrel®, and other thermoplastics.

In addition, adhesives can also be used to embed the fibers, so long as the adhesive is inert with respect to the chemical moieties attached to the fibers. Accordingly, mechanical adhesives can be used to embed fibers. Exemplary mechanical adhesives include rubbers, phenoxyresin, polysulfone, phenolics, urethanes, vinyl resins, acrylics, polyamides, polyesters, and the like. It is understood that the invention specifically excludes the use of chemical adhesives which can chemically react with the chemical moieties attached to the fibers since such chemical adhesives would not be inert with respect to the chemical moieties. However, in the case where cladding has been coated on the fibers, a chemical adhesive can be used since the adhesive would be prevented from contacting the attached chemical moieties, thereby protecting the chemical moieties from chemical modification by the adhesive. Exemplary chemical adhesives include epoxy, phenolic, polysulfide, polyurethane, silicones, polyester, resorcinolformaldehyde, cyanoacrylates, acrylates, and the like. It is understood that whether an adhesive is a mechanical or chemical adhesive can depend on the nature of the chemical moieties attached to the fibers. For example, if an adhesive can modify one particular attached chemical moiety but not a different chemical moiety, the adhesive is a chemical adhesive with respect to the first chemical moiety but a mechanical adhesive with respect to the different chemical moiety.

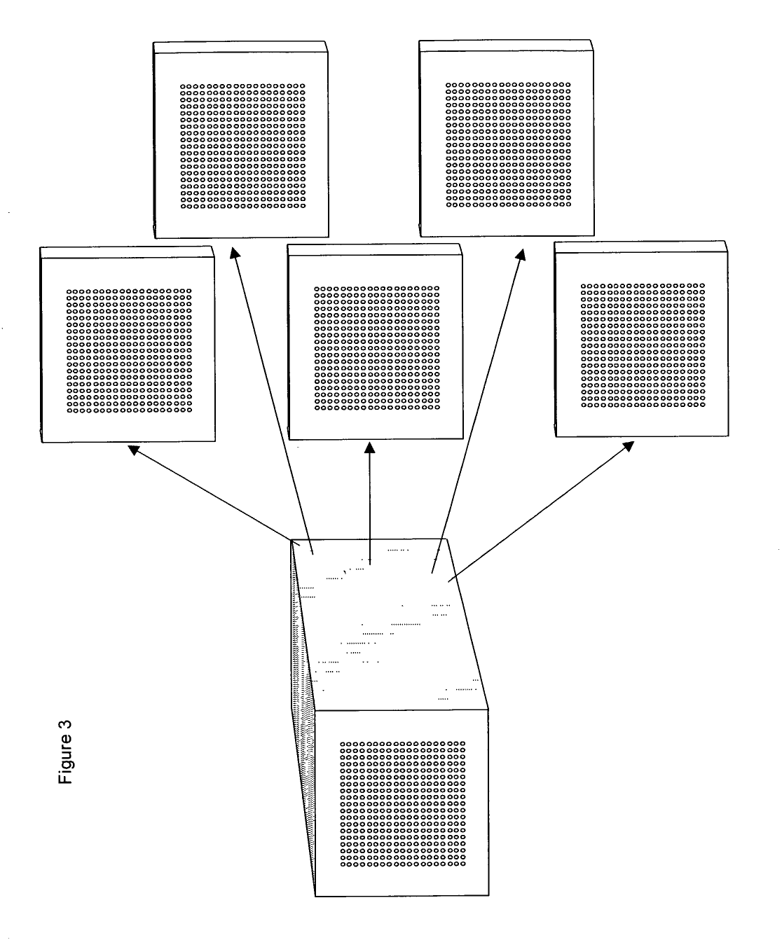

Once the fibers are embedded in the inert support, the embedded fibers can be sliced to generate substantially identical replicates. As shown in FIG. 3, the array can be sliced transversely to generate multiple biochips. The width of the slices can be varied depending on the needs of the particular application of using the chips. The slices are generally made at approximately 90° so that each slice contains a portion of each embedded fiber, although other angles can be used, if desired. The slices can be made so that less than all of the embedded fibers is included in the slice, although the slice will generally contain a portion of substantially all of the embedded fibers. The biochip face can be optionally polished, if desired.

After slicing the embedded fibers, very little of the surface attached molecules are exposed on the face of the biochip since the fibers are embedded. Therefore, limited interactions between the chemical moieties attached to the fiber and test molecules exposed to this biochip would be expected. Therefore, if desired, a portion of the embedding support can be removed to expose a non-end portion of the embedded fibers, resulting in an increase in surface area of the fibers having attached chemical moieties.

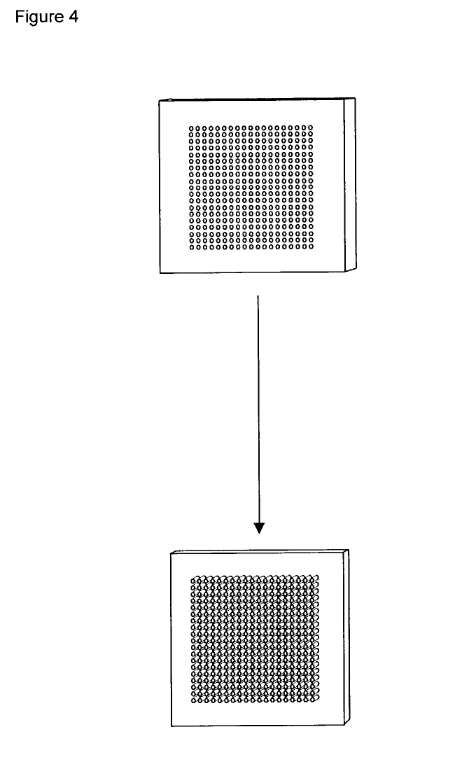

For example, the exposed surface area of each fiber can be increased as illustrated in FIG. 4 by dissolution of some of the inert support, leaving fibers protruding from the surface of the biochip and exposing a non-end portion of the fiber having attached molecules. The effective area available to interact with sample molecules for each of the protruding fibers follows the equation for a cylinder: Area=2πrh, where “r” is the radius of the fiber and “h” is the height of the exposed fiber above the surface of the embedding support. The surface area of each fiber probe would therefore exceed that of a 2-diminsional biochip when h/2>r. Fiber probes disclosed in the present invention would therefore be expected to yield higher sensitivity and higher density arrays than arrays produced by slicing biochips but not exposing additional surface area.

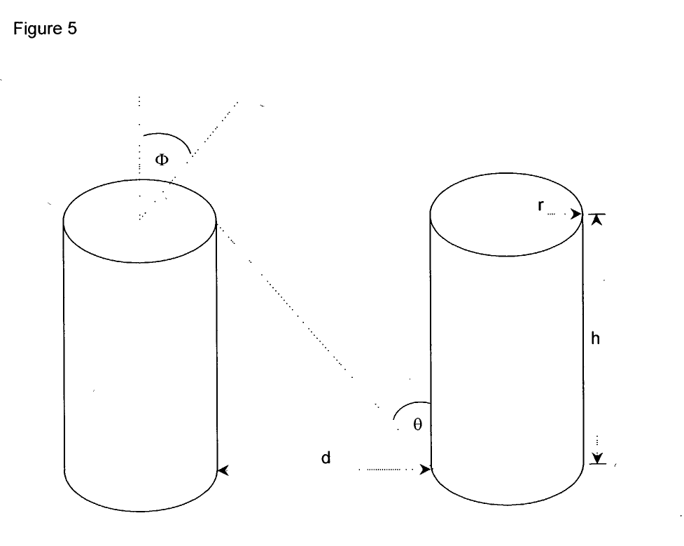

Light transmittance in an optical fiber is limited by the acceptance angle (Φ in FIG. 5). Light entering the optical fiber at an angle greater than the acceptance angle will not be transmitted in the fiber. In order to minimize crosstalk between two fibers, where fluorescent light emitted from one fiber enters an adjacent fiber at an angle (θ) less than the acceptance angle, the fibers should be spaced at a sufficient distance between fibers to minimize crosstalk. The distance between fibers (d) is therefore dependent upon the height of the fiber (h) protruding from the surface of the biochip such that

d>h/tan(90°−Φ)

For example, an array of fibers with acceptance angles of 60° and a height of 10 μm would require d>17.3 μm. With these dimensions, an array constructed from 10 μm fibers exposed 10 μm above the surface of the inert support would allow 134,000 elements per cm² while having a surface area of 314 μm² for each element.

Previously, the elimination of fiber crosstalk between adjacent fibers in a bundle has been detailed in U.S. Pat. No. 6,137,117, where multiple fibers or waveguides are aligned on an opaque or reflective support, rather than gathering fibers into a bundle. The separation of the fibers in this manner effectively removes the transfer of light from one fiber to another and, as a result, increases the optical resolution of the system.

In the present invention, the distance between the fibers such as waveguides or optical fibers can be set to minimize crosstalk between the fibers. A predetermined distance between fibers can be effected using a grid, as described in more detail above, or by coating with cladding, as described in more detail below. In particular, the spacing between the waveguides can be predetermined such that the spacing is greater than or equal to the height of an exposed non-end portion of the waveguide. If desired, the waveguides can be spaced at a distance that is greater than any desired height of the exposed non-end portion of the fiber, allowing the height of the exposed non-end portion to be varied for different applications but using the same spacing. It is understood that minimizing crosstalk means that the spacing between fibers is sufficient for the intended purpose. Thus, the spacing of the fibers can be such that crosstalk between fibers does occur so long as the amount of crosstalk does not interfere with the intended purpose of the array. If desired, the waveguides or optical fibers can be spaced such that essentially no crosstalk occurs between fibers.

In addition to using a grid to space the fibers at a predetermined distance, spacing of the fibers can also be effected by coating the fibers with cladding. Cladding is one or more layers of material of lower refractive index in intimate contact with a core material of higher refractive index. In a waveguide or optical fiber, the cladding surrounds the central core region, which supports guiding of the optical signal. For spacing the fibers at a predetermined distance, the cladding is coated at a sufficient thickness to space the fiber core at a predetermined distance when the cladded fibers are placed adjacent to each other. For example, the cladding can be coated at a thickness such that, when the cladded fibers are placed next to each other, the spacing between the core of adjacent fibers minimizes crosstalk between the fibers and can be spaced so that the spacing between the fibers is greater than or equal to the height of the exposed non-end portion of the fibers.

The invention also provides a method of generating an array of molecules by coating fibers with cladding. The method includes the steps of attaching one or more chemical moieties to a plurality of waveguides; coating each of the waveguides with cladding of sufficient thickness to space the waveguides at a predetermined distance; distributing the plurality of waveguides in a predetermined arrangement, thereby spacing the waveguides at a predetermined distance; fixing the plurality of waveguides in the predetermined arrangement; slicing the fixed plurality of waveguides transversely; and removing a portion of the cladding to expose a non-end portion of the fixed waveguides of at least one face of the slice, thereby generating an array of molecules.

Each optical fiber can be individually clad axially along its length. The cladding can be any material that has a lower refractive index, is opaque or reflective, and prevents the transmission of light energy photons from the optical fiber to the external environment or from the external environment into the fiber. The cladding can thus be composed of a variety of different chemical formulations, including silicones, plastics, cloths, platings and shielding matter of diverse chemical composition and formulation. Methods of cladding, including deposition, extrusion, painting and covering, are well known to those skilled in the art, and any of these known processes can be chosen to meet the requirements and convenience of the user. Cladding allows the alignment of fibers in an array without the use of additional spacers.

The use of clad waveguides or optical fibers also increases the options available for fixing the fibers together. For example, clad optical fibers can be fixed together using chemical adhesives as well mechanical adhesives and other inert supports for embedding fibers, as described above. Fixing of the clad waveguides or optical fibers functions similarly to the embedding of fibers described above in that the relative position of the waveguides or optical fibers is stabilized in a predetermined arrangement. This allows the fixed fibers to maintain the predetermined arrangement, even when being sliced.

The invention also provides a composition comprising a plurality of waveguides embedded in an inert support, the waveguides having attached thereto one or more chemical moieties and having a non-end portion exposed. The invention additionally provides a composition comprising a plurality of waveguides embedded in an inert support, the waveguides having attached thereto one or more chemical moieties and being spaced at a distance to minimize crosstalk between waveguides. The invention further provides a composition comprising a plurality of waveguides embedded in an inert support, the waveguides having attached thereto one or more chemical moieties, having a non-end portion exposed, and being spaced at a distance to minimize crosstalk between waveguides. The compositions can include a chemical moiety selected from the group consisting of nucleotide, nucleic acid, amino acid, polypeptide, and antibody. The waveguide can be a glass optical fiber.

The invention further provides methods of using the biochip arrays of molecules generated by the methods disclosed herein. For example, the biochips can be used for qualitative or quantitative assays of sample molecules. The sample is contacted with the biochip under conditions that allow specific binding of the sample molecules to the chemical moieties attached to the biochips. As used herein, specific binding means binding that is measurably different from a non-specific interaction. Specific binding can be measured, for example, by determining binding of a molecule compared to binding of a control molecule, which generally is a molecule of similar structure that does not have binding activity, for example, a peptide of similar size that lacks binding activity. Specificity of binding also can be determined, for example, by competition with a control molecule, for example, competition with an excess of the same molecule. In this case, specific binding is indicated if the binding of a molecule is competitively inhibited by itself. For example, specific binding between an antibody and antigen is measurably different from a non-specific interaction and occurs via the antigen binding site of the antibody. An antigen has binding activity for the antibody if the antibody specifically binds to the antigen.

As used herein, selective binding refers to a binding interaction that is both specific and discriminating between molecules, for example, an antibody that binds to a single molecule or closely related molecules. For example, an antibody can exhibit specificity for an antigen that can be both specific and selective for the antigen if the epitope is unique to a molecule. Thus, a molecule having selective binding can differentiate between molecules, as exemplified by an antibody having specificity for an epitope unique to one molecule or closely related molecules. Alternatively, an antibody can have specificity for an epitope that is common to many molecules, for example, a carbohydrate that is expressed on a number of molecules. Such an antibody has specific binding but is not selective for one molecule or closely related molecules.

As used herein, the term “sample” is intended to mean any biological fluid, body fluid, cell, tissue, organ or portion thereof, that includes one or more different molecules to be assayed. The term includes samples obtained or derived from an individual. For example, a sample can be a fluid sample such as body fluid, including blood, plasma, urine, saliva or sputum. A sample can also be a tissue section obtained by biopsy, cells that are placed in or adapted to tissue culture, or fractions or components purified or extracted from a biological fluid, tissue or cell. When using a cell or tissue sample, the sample can be processed to generate an extract that can be conveniently contacted with a biochip using methods well known to those skilled in the art. If desired, the sample can be prepared with denaturants, including detergents such as sodium dodecyl sulfate (SDS). The conditions for treating the sample can be chosen based on the desired type of assay.

When an assay is performed with a biochip made with a waveguide or optical fiber, the assay can be performed by monitoring changes in light transmission or emission based on binding of a sample molecule to a molecule attached to the fibers. For example, upon binding of one or more sample molecules to a biochip, changes in fluorescence of a fluorescently labeled sample molecule or fluorescently labeled chemical moiety attached to the waveguide or optical fiber can be monitored. If desired, the light monitoring can be performed on the opposite side of the biochip from where the sample molecules are bound by using the light transmission properties of the waveguide or optical fiber. Alternatively, changes can be monitored from the same side as the bound sample molecules.

Biochip microarrays such as the arrays disclosed herein can be used for a variety of purposes, including diagnostic purposes. The arrays can be used to identify disease related proteins or genes. For example, nucleic acid or oligonucleotide arrays can be used measure the expression level of genes or to identify unknown mutations (Ginot, Human Mutation 10:1-10 (1997)), sequencing by hybridization (Pease et al., Proc. Natl. Acad. Sci. USA 91:5022-5026 (1994)), and as biosensors (Liu et al., Anal. Biochem. 283:56-63 (2000)). Biochips for the analysis of proteins can be used for the purification of proteins, high throughput screening, microcapillary electrophoresis, protein profiling, and microsequencing (Figeys and Pinto, Electrophoresis 22:208-216 (2001)).

It is understood that modifications which do not substantially affect the activity of the various embodiments of this invention are also provided within the definition of the invention provided herein. Accordingly, the following examples are intended to illustrate but not limit the present invention.

### EXAMPLE I

### Generation of an Array by Embedding Fibers in an Inert Support

This example describes the generation of an array of fibers by distributing fibers in a grid and embedding the fibers in an inert support.

50 μm diameter glass optical fibers were formed into a 4×12 array by inserting fibers through two 1 cm² squares of 0.021″ stainless steel 200×200 mesh. The fibers were epoxied to one of the mesh squares. After the epoxy dried, the fibers were gently pulled taught, and the second mesh square was epoxied into position 10 cm from the first mesh square.

The fiber array was placed in a 12 cm×¾″×¾″ aluminum c-channel with ends sealed with aluminum foil. The wire mesh squares were fastened to the c-channel with steel wire such that the fiber array was held taught and did not touch the sides of the c-channel. Black polycarbonate pellets were placed below, beside, and above the fibers. The assembly was placed in an oven and heated to 450° F. for 3 hours. The assembly was removed from the oven and allowed to cool to room temperature before removing the fiber array now embedded in black polycarbonate. The polycarbonate rod was transversely sliced into 5 mm wide chips. The faces of the chips were polished using extra-fine wet emory cloth. One face of each of the chips was treated with alternating streams of methylene chloride and methanol forced from a Pasteur pipette. This solvent treatment resulted in the exposure of nominally 50 μm fiber ends from the surface of the chip.

This example demonstrates that generation of a fiber array by embedding in an inert support and slicing into replicate chips.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

I claim:

1. A method of generating an array of molecules, comprising the steps of:
(a) attaching one or more chemical moieties to a plurality of waveguides;
(b) distributing said plurality of waveguides in a predetermined arrangement in a grid, said grid thereby spacing said waveguides at a predetermined distance;
(c) embedding said plurality of waveguides in an inert support; and
(d) slicing said embedded plurality of waveguides transversely, thereby generating an array of molecules.

2. The method of claim 1, further comprising the step of removing a portion of said embedding inert support to expose a non-end portion of said embedded waveguides of at least one face of said slice.

3. The method of claim 1, wherein the distance between said waveguides minimizes crosstalk between waveguides.

4. The method of claim 3, wherein the spacing between said waveguides is greater than or equal to the height of said exposed non-end portion of said waveguides.

5. The method of claim 1, wherein said chemical moiety is selected from the group consisting of nucleotide, nucleic acid, amino acid, polypeptide, and antibody.

6. The method of claim 1, wherein said waveguide is a glass optical fiber.

7. A method of generating an array of molecules, comprising the steps of:
(a) attaching one or more chemical moieties to a plurality of waveguides;
(b) distributing said plurality of waveguides in a predetermined arrangement in a grid, said grid thereby spacing said waveguides at a predetermined distance, wherein the distance between said waveguides minimizes crosstalk between waveguides;
(c) embedding said plurality of waveguides in an inert support;
(d) slicing said embedded plurality of waveguides transversely; and
(e) removing a portion of said embedding inert support to expose a non-end portion of said embedded waveguides, thereby generating an array of molecules.

8. The method of claim 7, wherein the spacing between said waveguides is greater than or equal to the height of said exposed non-end portion of said waveguides.

9. The method of claim 6, wherein said chemical moiety is selected from the group consisting of nucleotide, nucleic acid, amino acid, polypeptide, and antibody.

10. The method of claim 7, wherein said waveguide is a glass optical fiber.

11. A method of generating an array of molecules, comprising the steps of:
(a) attaching one or more chemical moieties to a plurality of waveguides;
(b) coating each of said waveguides with cladding of sufficient thickness to space said waveguides at a predetermined distance;
(c) distributing said plurality of waveguides in a predetermined arrangement, thereby spacing said waveguides at a predetermined distance;
(d) fixing said plurality of waveguides in said predetermined arrangement;
(e) slicing said fixed plurality of waveguides transversely; and
(f) removing a portion of said cladding to expose a non-end portion of said fixed waveguides of at least one face of said slice, thereby generating an array of molecules.

12. The method of claim 11, wherein the distance between said waveguides minimizes crosstalk between waveguides.

13. The method of claim 12, wherein the spacing between said waveguides is greater than or equal to the height of said non-end portion of said waveguides.

14. The method of claim 11, wherein said chemical moiety is selected from the group consisting of nucleotide, nucleic acid, amino acid, polypeptide, and antibody.

15. The method of claim 11, wherein said waveguide is a glass optical fiber.

16. A composition comprising a plurality of waveguides embedded in an inert support, said waveguides having attached thereto one or more chemical moieties and having a non-end portion exposed.

17. The composition of claim 16, wherein said chemical moiety is selected from the group consisting of nucleotide, nucleic acid, amino acid, polypeptide, and antibody.

18. The composition of claim 16, wherein said waveguide is a glass optical fiber.

19. A composition comprising a plurality of waveguides embedded in an inert support, said waveguides having attached thereto one or more chemical moieties and being spaced at a distance to minimize crosstalk between waveguides.

20. The composition of claim 19, wherein said chemical moiety is selected from the group consisting of nucleotide, nucleic acid, amino acid, polypeptide, and antibody.

21. The composition of claim 20, wherein said waveguide is a glass optical fiber.

22. A composition comprising a plurality of waveguides embedded in an inert support, said waveguides having attached thereto one or more chemical moieties, having a non-end portion exposed, and being spaced at a distance to minimize crosstalk between waveguides.

23. The composition of claim 22, wherein said chemical moiety is selected from the group consisting of nucleotide, nucleic acid, amino acid, polypeptide, and antibody.

24. The composition of claim 22, wherein said waveguide is a glass optical fiber.
